(19) United States
(12) Patent Application Publication
(10) Pub. No.: US 2025/0352146 A1
(43) Pub. Date: Nov. 20, 2025

(54) Medical Imaging Apparatus with a Patient Support Apparatus

(71) Applicant: Siemens Healthineers AG, Forchheim (DE)

(72) Inventors: Stefanie Gügel-Wild, Langensendelbach (DE); Florian Meise, Hausen (DE)

(73) Assignee: Siemens Healthineers AG, Forchheim (DE)

(21) Appl. No.: 19/207,746

(22) Filed: May 14, 2025

(30) Foreign Application Priority Data
May 15, 2024 (DE) 10 2024 204 482.2

(51) Int. Cl. A61B 5/00; A61B 5/055

(52) U.S. Cl. CPC A61B 5/704; A61B 5/055

(57) ABSTRACT

The disclosure is directed to a medical imaging apparatus with a scanner unit, a patient receiving region that is at least partially surrounded by the scanner unit, and a patient support apparatus. The patient support apparatus comprises a patient chair, which has at least two different support regions, and an adjusting unit. A first support region of the at least two support regions comprises a first support area, and a second support region of the at least two support regions comprises a second support area, and the adjusting unit is configured for an adjustment of an orientation of the first support area and for an adjustment of an orientation of the second support area.

### CROSS-REFERENCE TO RELATED APPLICATIONS

The present application claims priority to and the benefit of Germany patent application no. DE 10 2024 204 482.2, filed on May 15, 2024, the contents of which are incorporated herein by reference in their entirety.

### TECHNICAL FIELD

The present disclosure relates to a medical imaging apparatus having a scanner unit, a patient receiving region at least partially surrounded by the scanner unit, and a patient support apparatus.

### BACKGROUND

In medical imaging, the positioning of a patient and the correct positioning of accessory units are of decisive importance for a high image quality that is needed as the basis for a correct diagnosis. In particular, in magnetic resonance imaging examinations, apart from the correct positioning of the patient, a correct positioning of local radio frequency coils that are arranged around the region of the patient to be examined to acquire magnetic resonance signals are of great significance for high image quality.

For a correct positioning, the patient, in particular the region of the patient to be examined, should be positioned within an isocenter of the medical imaging apparatus, in particular the scanner unit of the medical imaging apparatus. For the positioning of the region to be examined in the isocenter of the scanner unit, in conventional medical imaging apparatuses, for example a magnetic resonance apparatus, a computed tomography apparatus, a PET apparatus, etc., a patient support apparatus with a movable patient table is available. By means of the movable patient table, the patient, in particular the region of the patient to be examined, is moved into a patient receiving region of the medical imaging apparatus.

However, a positioning on the patient table presents a challenge for many patients. Currently, the patients sit down on the patient table or are placed sitting on the patient table and must then themselves lie down or be laid down, preferably after a 90° rotation. The same procedure naturally applies in the same way for dismounting from the patient table so that the patient can leave the room.

However, different patients have differing heights and statures, which can contribute, in some patients, to problems in the positioning. The patient tables are configured to be height-adjustable and are lowered for the seating of the patient on the patient table. However, the physical constitution of patients, for example older patients and/or disabled patients, can also play a role in the positioning so that a time-consuming positioning of the patient is necessary.

If, in addition, special measurements are carried out for which the patients must adopt a particular position, such as for example laying on the abdomen, this requires an additional positioning effort for both the patient and also for the medical operating personnel.

### SUMMARY

It is an object of the present disclosure to provide a simple and time-saving positioning of a patient for a medical imaging examination, e.g. a magnetic resonance examination. This object is achieved with the features of the embodiments as discussed herein, including the claims.

The present disclosure relates to a medical imaging apparatus having a scanner unit, a patient receiving region at least partially surrounded by the scanner unit, and a patient support apparatus. According to the disclosure, the patient support apparatus comprises a patient chair which has at least two different support regions and an adjusting unit, wherein a first support region of the at least two support regions comprises a first support area and a second support region of the at least two support regions comprises a second positioning area and the adjusting unit is configured for an adjustment of an orientation of the first support area and for an adjustment of the orientation of the second support area.

The medical imaging apparatus can comprise, for example, a computed tomography (CT) apparatus, a positron emission tomography (PET) apparatus, an X-ray apparatus, a magnetic resonance apparatus, any combination thereof, etc. The medical imaging apparatus may e.g. comprise a magnetic resonance apparatus since, for magnetic resonance examinations, a correct positioning of the patient and of local radio frequency coils that are arranged around the region of the patient to be examined for acquiring magnetic resonance data is necessary. In addition, due to the relatively long examination time in magnetic resonance examinations, an incorrect positioning may result in even additional delays.

The medical imaging apparatus may e.g. comprise a medical and/or diagnostic medical imaging apparatus that is constructed and/or configured for acquiring medical and/or diagnostic image data of a patient. In an embodiment, the scanner unit of the medical imaging apparatus is configured for acquiring the medical and/or diagnostic image data. If, for example, the medical imaging apparatus is configured as a magnetic resonance apparatus, the scanner unit can comprise a magnet unit with a main magnet for generating a homogeneous magnetic field, a gradient system for position encoding the acquired magnetic resonance data, and a radio frequency antenna unit. However, if the medical imaging apparatus is configured as a CT apparatus, the scanner unit may alternatively or additionally comprise an X-ray detector.

For a medical imaging examination, for example a magnetic resonance examination, the patient, e.g. the region of the patient to be examined, is positioned within a patient receiving region of the medical imaging apparatus. The field of view (FOV) and/or an isocenter of the medical imaging apparatus may then be arranged within the patient receiving region. The FOV may e.g. comprise an acquisition region of the medical imaging apparatus within which the conditions exist for an acquisition of medical image data within the patient receiving region. For example, in one embodiment of the medical imaging apparatus as a magnetic resonance apparatus, the FOV may comprise a homogeneous main magnetic field. The isocenter of the medical imaging apparatus may e.g. comprise the region and/or point within the medical imaging apparatus having optimal and/or ideal conditions for the acquisition of medical image data. For example, in one embodiment of the medical imaging apparatus as a magnetic resonance apparatus, the isocenter may comprise the most homogeneous magnetic field region within the scanner unit.

For a movement and/or positioning of the patient, e.g. of the region of the patient to be examined within the patient receiving region, the medical imaging apparatus comprises a patient support apparatus. Therein, the patient support apparatus has a base unit and the patient chair. The patient chair is configured to be movable relative to the scanner unit and/or the base unit of the patient support apparatus. In an embodiment, the patient chair is configured such that it is able to be introduced and/or moved within the patient receiving region in the longitudinal direction of the patient receiving region to position the patient in an examination position within the patient receiving region.

The patient chair also has at least two different support regions. The different support regions of the patient chair may e.g. be constructed and/or configured for supporting and/or positioning different body regions of patients. In addition, the patient support apparatus has an adjusting unit that is configured for an adjustment of an orientation of the first support area of the first support region and of the second support area of the second support region. The adjusting unit may comprise e.g. at least one adjusting element for each support region for adjusting the orientation of each support area. In an embodiment, the adjusting unit also comprises a control unit, by means of which a control of a setting of the individual support regions, e.g. an adjustment of an orientation of the individual support areas, can take place. In an embodiment, for adjusting the orientation of the first support area and/or of the second support area, the two support regions are configured such that they are able to move relative to one another. Therein, for adjusting the orientation of the first support area and/or of the second support area, the two support regions are configured such that they are able to move in different directions. In addition, for adjusting the orientation of the first support area and/or of the second support area, the two support regions are also able to move in different directions simultaneously. For a movement of the patient chair into the patient receiving region, the individual support regions of the patient chair are situated in an introducing position.

The first support region and the second support region may e.g. be arranged by adjoining one another so that a continuous support area for supporting the patient can be provided by means of the two support regions. The orientation of the first support area may e.g. comprise an inclination with any suitable inclination angle, e.g. between 0° and 90°. Therein, the orientation of the first support area with an inclination angle of 0° may comprise an orientation in a horizontal and/or level direction and, with an inclination angle of 90°, an orientation in a vertical and/or perpendicular direction. The orientation of the second support area may comprise an inclination with any suitable inclination angle, e.g. between 0° and 90°. Therein, the orientation of the second support area with an inclination angle of 0° can comprise an orientation in a horizontal and/or level direction and with an inclination angle of 90°, an orientation in a vertical and/or perpendicular direction.

The disclosure has the advantage that a simple and time-saving positioning of a patient on a patient support apparatus for a medical imaging examination, for example a magnetic resonance examination, can be enabled. In an embodiment, by way of an adjustment of the different support regions, e.g. the orientation of the support areas of the different support regions, a simple seating and positioning of a patient on the patient support apparatus, e.g. the patient chair, can be achieved. Herein, e.g. small patients, relatively old patients, patients with a disability, etc. may advantageously be supported by changing the orientation of the individual support areas of the individual support regions when being seated and positioned on the patient support apparatus.

In an advantageous development of the medical imaging apparatus according to the disclosure, it can be provided that the first support region of the at least two support regions comprises a seat region of the patient chair and the second support region of the at least two support regions is configured for a support of an upper body of a patient. In this way, a simple positioning of a patient can be enabled. In an embodiment, for a seating of the patient, the seat region, e.g. the first support area, can have a level orientation and/or a horizontal orientation and the support region configured for supporting an upper body of a patient with the second support region can have an inclination to the second support area of any suitable angle or range of angles, such as e.g. between 70° and 90°, between 75° and 90°, between 80° and 90°, etc. For a positioning and/or preparation of the patient for the upcoming medical imaging examination, an inclination of the second support area of the second support region, e.g. the support region configured for supporting an upper body of a patient, may be brought into a level orientation.

This embodiment offers an advantageous support during seating and laying of the patient into an examination position on the patient support apparatus. In addition, in this way, an advantageous support can be provided for a patient during a dismounting from and/or leaving of the patient support apparatus. For instance, for patients with physical disabilities, a comfortable positioning and/or laying and/or dismounting can thus be provided.

In an advantageous development of the medical imaging apparatus according to the disclosure, it can be provided that the patient chair comprises at least one further support region, wherein the at least one further support region is configured for supporting a head of a patient and wherein the at least one further support region has a further support area and the adjusting unit is configured for an adjustment of an orientation of the further support area. The further support region may e.g. comprise a third support region with a third support area. This third support region may e.g. be arranged adjoining the second support region for supporting an upper body of a patient. In an embodiment, the second support region is arranged between the first support region and the third support region. The orientation of the third support area can therein comprise an inclination with any suitable inclination angle, e.g. between 0° and 90°. Therein, the orientation of the third support area can comprise, with an inclination angle of 0°, an orientation in a horizontal and/or level direction and, with an inclination angle of 90°, an orientation in a vertical and/or perpendicular direction.

This embodiment offers an advantageous support and/or positioning of a head of a patient during a medical imaging examination. In an embodiment, the third support region may also be adjusted such that an inclination of the third support area can be adapted to the patient and/or to the medical imaging examination.

In an advantageous development of the medical imaging apparatus according to the disclosure, it can be provided that the patient chair comprises at least one further support region, wherein the at least one further support region is configured for supporting the lower legs and/or feet of a patient and wherein the at least one further support region has a further support area and the adjusting unit is configured for an adjustment of an orientation of the further support area. The further support region may e.g. comprise a fourth support region with a fourth support area. This fourth support region may e.g. be arranged adjoining the first support region with the first support area configured as a seat area. In an embodiment, the first support region is arranged between the second support region and the fourth support region. The orientation of the fourth support area can therein comprise an inclination with an inclination angle of any suitable angle, e.g. between 0° and 90°. Therein, the orientation of the fourth support area with an inclination angle of 0° can comprise an orientation in a horizontal and/or level direction and, with an inclination angle of 90°, an orientation in a vertical and/or perpendicular direction.

This embodiment offers an advantageous supporting and/or positioning of a patient during a medical imaging examination. In an embodiment, by means of the further support region that is configured for a supporting of lower legs and/or feet of a patient, a further support region is available to the patient chair and thus different support regions for a support and/or positioning of different body regions of patients. In an embodiment, a patient chair with four different support regions enables a comfortable supporting and/or positioning of the entire patient.

In an embodiment, the four support regions with the four support areas form a continuous support area for receiving and/or supporting the patient.

In an advantageous development of the medical imaging apparatus according to the disclosure, it can be provided that the patient chair comprises a laying mode, wherein in the laying mode, the adjusting unit positions the individual support regions of the patient chair into a laying position and, in the laying mode, the patient chair can be moved into the patient receiving region. A selection of the laying mode can be undertaken by a user, wherein an adjustment of the individual support regions into the laying position may e.g. occur automatically by means of the adjusting unit. In an embodiment, the laying mode of the patient chair comprises a defined position and orientation of the individual support regions of the patient chair. In the laying position, the individual support regions are in a level and/or horizontal position with level and/or horizontally oriented support areas. In the laying mode, the at least two support regions of the patient chair are in the laying position with level and/or horizontally oriented support areas. In an embodiment, in the laying mode of the patient chair, all four support regions of the patient chair are in a level and/or horizontal position with level and/or horizontally oriented support areas. In the laying mode of the patient chair, the individual support regions and thus the individual support areas of the patient chair are in an introducing position for a movement into the patient receiving region of the scanner unit. In an embodiment, the at least two support areas, e.g. all four support areas, may form a continuous laying area for the patient, wherein the individual support areas are arranged at the same height.

In this way, the patient chair may be arranged particularly rapidly and simply into the laying mode by means of the adjusting unit. A further advantage is that the laying mode provides a space-saving position for a movement into the patient receiving region. In addition, by way of the laying mode, e.g. the fact that the individual support areas are arranged at the same height, a contact of the patient with a housing of the scanner unit surrounding the patient receiving region during a medical imaging examination can advantageously be prevented. For instance, during magnetic resonance examinations, for example, a contact between the patient and the housing surrounding the patient receiving region is to be prevented.

In an advantageous development of the medical imaging apparatus according to the disclosure, it can be provided that the patient chair comprises a sitting mode, wherein in the sitting mode, the adjusting unit positions the individual support regions of the patient chair into a sitting position. Therein, a selection of the sitting mode can be undertaken by a user, wherein an adjustment of the individual support regions into a sitting position may e.g. occur automatically by means of the adjusting unit. In an embodiment, the sitting mode of the patient chair comprises a defined position and orientation of the individual support regions of the patient chair. In the sitting position, the different support regions have different orientations of their support areas. In an embodiment, the support region of the patient chair configured as a seat region, e.g. the first support region, is herein arranged in a level and/or horizontal position with a level and/or horizontally oriented support area. In an embodiment, the support region configured for supporting an upper body of a patient is herein arranged in a substantially perpendicular and/or vertical position with a substantially perpendicularly and/or vertically oriented support area. In addition, the further support regions, e.g. the support region configured for supporting a head of the patient and/or for supporting lower legs and/or feet of a patient, are arranged in a substantially perpendicular and/or vertical position with a substantially perpendicularly and/or vertically oriented support area. A substantially perpendicular and/or vertical position with a substantially perpendicularly and/or vertically oriented support area can have an inclination angle, in relation to the level and/or horizontal position with a level and/or horizontally oriented support area, of any suitable angle or range of angles, such as e.g. between 75° and 90°, between 80° and 90°, etc.

This embodiment of the disclosure has the advantage that the patient chair can be arranged particularly rapidly and simply into the sitting mode by means of the adjusting unit. In this way, a simple seating of the patients onto the patient chair can be achieved. Moreover, patients can also be supported during positioning from a laying position into the sitting position after a medical imaging examination.

In an advantageous development of the medical imaging apparatus according to the disclosure, it can be provided that the patient chair comprises a prone laying mode, wherein in the prone laying mode, the adjusting unit positions the individual support regions of the patient chair into a prone laying position. A selection of the prone laying mode can be undertaken by a user, wherein an adjustment of the individual support regions into a prone laying position may e.g. take place automatically by means of the adjusting unit. In an embodiment, the prone laying mode of the patient chair comprises a defined position and orientation of the individual support regions of the patient chair. In the prone laying position, the different support regions have different orientations of their support areas. In an embodiment, the support region configured for supporting lower legs and/or feet of a patient, e.g. the fourth support region, is herein arranged in a level and/or horizontal position with a level and/or horizontally oriented support area so that a patient can kneel thereon. In an embodiment, the remaining support regions of the patient chair, e.g. the support region configured as a seat region of the patient chair and the support region configured for supporting an upper body of a patient, are arranged in a substantially perpendicular and/or vertical position with a substantially perpendicularly and/or vertically oriented support area. In addition, the further support region, e.g. the support region (where present) configured for supporting a head of the patient, is arranged in a substantially perpendicular and/or vertical position with a substantially perpendicularly and/or vertically oriented support area. A substantially perpendicular and/or vertical position with a substantially perpendicularly and/or vertically oriented support area can have an inclination angle, in relation to the level and/or horizontal position with a level and/or horizontally oriented support area, of any suitable angle or range of angles such as e.g. between 75° and 90°, between 80° and 90°, etc.

This embodiment of the disclosure has the advantage that a simple positioning of a patient into a prone laying position can be enabled. For instance, the patient may kneel on the patient chair on the support region configured for supporting lower legs and/or feet of a patient and can lean with their front side on the remaining support regions. Thereafter, the patient chair can be brought into the laying mode, by means of the adjusting unit, for movement into the patient receiving region. For example, for relatively old patients and/or disabled patients, the flexible adjustment of the individual support regions offers a secure positioning in a prone position on the patient chair.

In an advantageous development of the medical imaging apparatus according to the disclosure, it can be provided that the adjusting unit has a control unit, wherein the control unit is configured to carry out a separate actuation of the individual support regions for adjusting an orientation of each support area. A separate actuation of the individual support regions should be understood herein to mean that an orientation of a support area of one of the support regions is adjustable independently of an orientation of a support area of one of the further support regions. In this way, an individual adjustment of an orientation of the individual support regions, for example an adjustment of the orientation of the individual support regions adapted to a patient, can advantageously be achieved. For example, the adjustment for the orientation of the individual support regions can differ for patients of different height. In addition, the adjustment of the orientation of the individual support regions can be adapted to a medical imaging examination.

Alternatively to the configuration of the adjusting unit with a control unit, e.g. a single control unit, the adjusting unit can also have a separate control unit for each support region of the patient chair.

The control unit comprises at least one computation module and/or a processor. Thus, the control unit is configured, for example, to carry out computer-readable instructions. In an embodiment, the control unit comprises a memory storage unit, wherein computer-readable information is stored on the memory storage unit, and wherein the control unit is configured to load the computer-readable information from the memory storage unit and to execute the computer-readable information. In this way, the control unit according to the disclosure is configured to carry out an actuation of the individual support regions, e.g. a separate actuation of the individual support regions for adjusting an orientation of each support area.

The components of the control unit may be configured primarily in the form of software components. In principle, these components may however also be realized, e.g. if especially rapid calculations are needed, at least partially in the form of software-supported hardware components, for example FPGAs or suchlike. Similarly, the required interfaces can be configured, for example if only the accepting of data from other software components is needed, as software interfaces. However, these may also be designed as interfaces which are configured as hardware and are actuated by way of suitable software. It is naturally also conceivable that a plurality of the aforementioned components are implemented grouped together in the form of a single software component and/or a software-supported hardware component.

In an advantageous development of the medical imaging apparatus according to the disclosure, it can be provided that the patient support apparatus has a base unit with a vertical adjusting unit, wherein the vertical adjusting unit is configured for an adjustment of a vertical position of the patient chair. The vertical adjusting unit may comprise, for example, a lifting unit and/or a scissor lift and/or other units deemed useful by a person skilled in the art for vertical positioning of the patient chair. By means of the vertical adjusting unit, an adjustment of a height and/or a vertical position of the entire patient chair takes place. In this way, by lowering the patient chair, an easy seating of the patient onto the patient chair and/or an easy dismounting of the patient from the patient chair is enabled. In addition, by raising the patient chair, it can be placed in an introducing position for moving into the patient receiving region. In an embodiment, the patient chair is situated in a higher position in the introducing position than in the seating position.

In an advantageous development of the medical imaging apparatus according to the disclosure, it can be provided that the patient support apparatus comprises a rotary unit, wherein the rotary unit is configured for an adjustment of an orientation of the patient chair relative to the scanner unit and/or relative to a base unit of the patient support apparatus. The rotary unit can be included by the base unit of the patient support apparatus. In an embodiment, the rotary unit is configured to rotate the patient chair about a perpendicular and/or vertical axis. For instance, the rotary unit may be configured to rotate the patient chair as a whole about a perpendicular and/or vertical axis. This embodiment of the disclosure enables a simple positioning and/or seating onto the patient chair for a medical imaging examination in which the patient is moved feet first into the patient receiving region. In an embodiment, the patient can sit down on the patient chair with their back to the scanner unit or on the patient chair rotated by 90° relative to the introducing position into the patient receiving region. This enables a simple seating onto the patient chair since a restriction by the scanner unit during seating is advantageously prevented. In an embodiment, following seating onto the patient chair, the patient chair is rotated by means of the rotary unit into the examination position, wherein in an examination position in which the patient is moved feet first into the patient receiving region, the patient chair is rotated such that after the rotation, the patient is looking at the scanner unit and/or the patient is turned with their face toward the scanner unit. By contrast, in an examination position in which the patient is moved head first into the patient receiving region, the patient chair is rotated such that after the rotation, the patient is turned with their back toward the scanner unit.

In an advantageous development of the medical imaging apparatus according to the disclosure, it can be provided that at least one support region of the at least two support regions has a receiving region for receiving an accessory unit. The receiving region for receiving the accessory unit can therein be arranged below the support area of the support region. In an embodiment, each of the support regions of the patient chair has its own receiving region for receiving an accessory unit. The individual receiving regions advantageously each have an opening, wherein the openings are each arranged on a side region of the patient chair and thus on a side region of the respective support region, so that a simple introduction of the accessory unit or a simple removal of the accessory unit can be enabled. For example, the accessory unit can comprise a control unit of an ECG unit, a weight sensor acquisition unit, and/or further accessory units (also referred to herein simply as accessories) deemed useful by a person skilled in the art. If the medical imaging apparatus is configured as a magnetic resonance apparatus, the accessory unit can also comprise a local radio frequency coil that is configured for acquiring magnetic resonance data.

This embodiment of the disclosure enables a particularly space-saving arrangement of the accessory unit during the medical imaging examination. For example, by way of such an arrangement of the accessory unit, a restriction of space and/or room available for the patient during the medical imaging examination can advantageously be prevented.

In an advantageous development of the medical imaging apparatus according to the disclosure, it can be provided that the patient support apparatus comprises a stationary patient support apparatus. The stationary patient support apparatus may e.g. have a fixed position in relation to the scanner unit. In an embodiment, the base unit of the patient support apparatus has a fixed position in relation to the scanner unit. By contrast, the patient chair is rotatable and is adjustable in its height and is configured for a movement into the patient receiving region and can therein be moved relative to the base unit and/or the scanner unit. In an embodiment, apart from a vertical adjusting unit and a rotary unit, a horizontal adjusting unit, by means of which a movement of the patient chair into the patient receiving region takes place, is also arranged within the base unit. A stationary patient support apparatus has the advantage that a time-consuming docking of the patient support apparatus for a medical imaging examination can advantageously be dispensed.

In an advantageous development of the medical imaging apparatus according to the disclosure, it can be provided that the patient support apparatus comprises a mobile patient support apparatus with a coupling unit, wherein the coupling unit is compatible with a coupling unit of the scanner unit. The mobile patient support apparatus may e.g. have a chassis with a plurality of wheels for a movement of the patient support apparatus in the room. For a coupling to the scanner unit, the patient support apparatus has the coupling unit that is compatible with a coupling unit of the scanner unit. By means of the coupling unit, a mechanical coupling of the patient support apparatus to the scanner unit can take place, wherein the mechanical coupling positions the patient support apparatus in a correct position in relation to the scanner unit and thus for a movement into the patient receiving region. Alternatively or additionally, the coupling by means of the coupling unit may comprise an electrical and/or an electronic coupling of the patient support apparatus to the scanner unit.

In an advantageous development of the medical imaging apparatus according to the disclosure, it can be provided that the medical imaging apparatus comprises a magnetic resonance apparatus and the patient table is configured to be compatible with magnetic resonance.

### BRIEF DESCRIPTION OF THE DRAWINGS

Further advantages, features and details of the disclosure are disclosed in the exemplary embodiment described below making reference to the drawings.

In the drawings:

FIG. 1 illustrates a schematic representation of an example medical imaging apparatus with an example patient chair, according to embodiments of the present disclosure;

FIG. 2A-2C illustrate a transition of an example patient chair from a sitting mode into a laying mode, according to embodiments of the present disclosure;

FIG. 3A-3C illustrate a transition of the example patient chair from a prone laying mode into the laying mode, according to embodiments of the present disclosure;

FIG. 4 illustrates a movement of the example patient chair in the laying mode into a patient receiving region of the medical imaging apparatus, according to embodiments of the present disclosure; and

FIG. 5A-5B illustrate the example medical imaging apparatus with an example mobile patient support apparatus, according to embodiments of the present disclosure.

### DETAILED DESCRIPTION OF THE DISCLOSURE

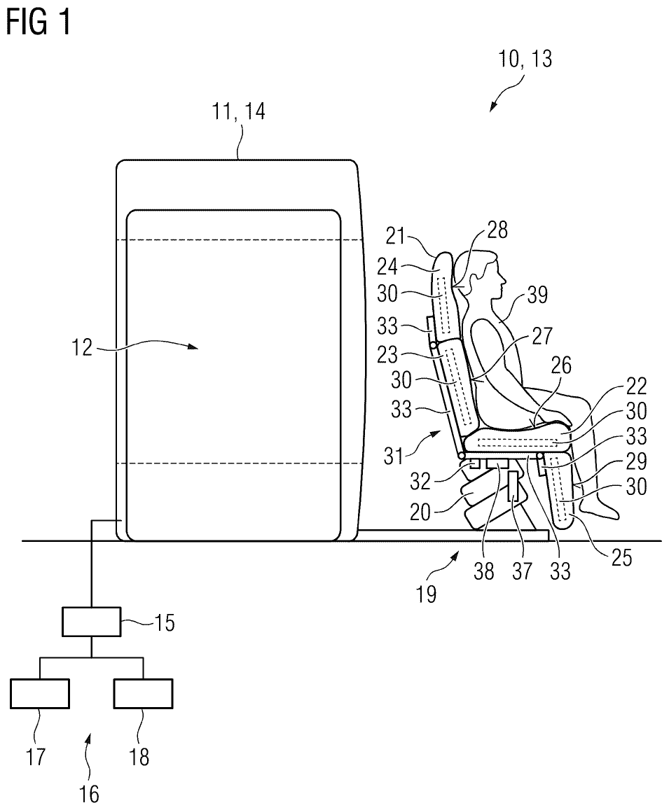

FIG. 1 illustrates a schematic representation of an example medical imaging apparatus with an example patient chair, according to embodiments of the present disclosure. Specifically, FIG. 1 shows a schematic representation of a medical imaging apparatus 10. The medical imaging apparatus 10 comprises a scanner unit (also referred to herein as a scanner) 11 and a patient receiving region 12 surrounded at least partially by the scanner unit 11. In the present exemplary embodiment, the patient receiving region 12 is configured to be cylindrical and is surrounded cylindrically in a circumferential direction by the scanner unit 11. In principle, however, an embodiment of the patient receiving region 12 deviating therefrom is readily conceivable.

In the present exemplary embodiment, the medical imaging apparatus 10 is formed by a magnetic resonance apparatus 13, wherein by way of example the present disclosure is described on the basis of the magnetic resonance apparatus 13. The present disclosure is, however, not restricted to the embodiment of the medical imaging apparatus 10 as a magnetic resonance apparatus 13, and further embodiments of the medical imaging apparatus 10 are readily conceivable, for example an embodiment as a CT apparatus or as a PET apparatus, etc. The magnetic resonance apparatus 13 comprises a scanner unit 11 configured as a magnet unit 14 with a main magnet, a gradient coil unit and a radio frequency antenna unit.

For control of the medical imaging apparatus 10, it has a system control unit 15 (also referred to herein as a system controller or simply a controller). By means of the system control unit 15, an execution of a medical imaging examination on a patient can be controlled. For this purpose, the system control unit 15 can have a control software and a processor. In addition, the system control unit 15 can also comprise a memory storage unit for storing the control software. For an interaction with a user of the medical imaging apparatus 10, it has a user interface 16 (also referred to herein as a user interface) with an input unit 17 (also referred to herein as an input interface) and an output unit 18 (also referred to herein as an output interface).

For a positioning of the patient 39, e.g. a region of the patient 39 that is to be examined, within the patient receiving region 12, the medical imaging apparatus 10, e.g. the magnetic resonance apparatus 13, has a patient support apparatus 19 (also referred to herein as a patient support). The patient support apparatus 19 herein comprises a stationary patient support apparatus 19 that is permanently connected to the scanner unit 11. The patient support apparatus 19 has a base unit (also referred to herein as a base) 20 and a patient chair 21 that is movable relative to the base unit 20. For a positioning of the patient 39, e.g. the region of the patient 39 that is to be examined, the patient chair 21 is configured to be movable within the patient receiving region 12. In an embodiment, the patient chair 21 is herein mounted to be movable in the direction of a longitudinal extent of the patient receiving region 12 and/or in the z-direction.

The patient chair 21 has at least two different support regions 22, 23, 24, 25. In the present exemplary embodiment, the patient chair 21 has four different support regions 22, 23, 24, 25. Therein, a first support region 22 comprises a seat region of the patient chair 21 with a first support area 26, e.g. a seat area. A second support region 23 of the patient chair 21 is configured for a support of an upper body of a patient 39 and comprises a second support area 27. A third support region 24 of the patient chair 21 is configured for a support of a head of a patient 39 and comprises a third support area 28. A fourth support region 25 of the patient chair 21 is configured for a support e.g. of lower legs and/or feet of a patient 39 and comprises a fourth support region 29.

The individual support regions 22, 23, 24, 25 of the patient chair each have a receiving region 30 for receiving an accessory unit (also referred to herein as an accessory). In the present exemplary embodiment, the individual receiving regions 30 are configured such that they are formed for receiving a local radio frequency coil for a magnetic resonance examination. Therein, the individual receiving regions 30 may also comprise any suitable connector, such as e.g. a connection socket for coupling the local radio frequency coil for a data exchange with the local radio frequency coils.

Furthermore, the patient chair 21 has an adjusting unit 31 (also referred to herein as an adjuster), which is configured for adjusting an orientation of the individual support areas 26, 27, 28, 29 of the individual support regions 22, 23, 24, 25. For this purpose, each adjusting unit 31 may e.g. have at least one adjusting element 33 for adjusting the orientation of each support area 26, 27, 28, 29. In addition, the adjusting unit 31 may have a control unit 32 (also referred to herein as a controller) for controlling the adjustment of the orientation of the support areas 26, 27, 28, 29. The control unit 32 may be configured, for instance, for a separate actuation of the individual support regions 22, 23, 24, 25 for adjusting an orientation of each support area 26, 27, 28, 29. For this purpose, the control unit 32 has a corresponding control software and/or control programs which are stored in a memory storage unit. The memory storage unit can therein be included by the control unit 32. Alternatively thereto, the memory storage unit can also be configured separately from the control unit 32. The control unit 32 may also e.g. have a processor, which is configured for an execution of the control software and/or control programs, wherein on an execution of the control software and/or control programs, an adjustment of an orientation of a support area 26, 27, 28, 29 of a support region 22, 23, 24, 25 or an adjustment of an orientation of a plurality of support areas 26, 27, 28, 29 of a plurality of support regions 22, 23, 24, 25 of the patient chair 21 is carried out.

The base unit 20 of the patient support apparatus 19 has a vertical adjusting unit 37 (also referred to herein as a vertical adjuster), wherein the vertical adjusting unit 37 is configured for an adjustment of a vertical position of the patient chair 21. For example, for a seating of a patient 39 onto the patient chair 21 or for a dismounting of a patient 39 from the patient chair 21, the patient chair 21 may be placed in a low position by means of the vertical adjusting unit 37. However, for a movement of the patient chair 21 into the patient receiving region 12, the patient chair 21 may be moved by means of the vertical adjusting unit 37 into a higher position in which a movement into the patient receiving region 12 is possible. The base unit 20 of the patient support apparatus 19 further comprises a rotary unit 38 (also referred to herein as a rotator), wherein the rotary unit 38 is configured for an adjustment of an orientation of the patient chair 21 relative to the scanner unit 11 and/or relative to the base unit 20. The rotary unit 38 may e.g. be configured for an adjustment of an orientation of the patient chair 21 as a whole. In an embodiment, the rotary unit 38 is configured for a rotation of the patient chair 21 in a horizontal plane.

The patient chair 21 comprises different chair modes in which the individual support areas 26, 27, 28, 29 have a defined orientation and/or position. The different chair modes can therein be selected by a user, for example at the user interface 16 of the medical imaging apparatus 10 or at a user interface (not shown) of the patient support apparatus 19. An adjustment of a selected chair mode then may e.g. take place automatically by means of the adjusting unit 31, wherein the adjusting unit 31 positions the individual support regions 22, 23, 24, 25 in a position defined and/or characterized for the selected chair mode.

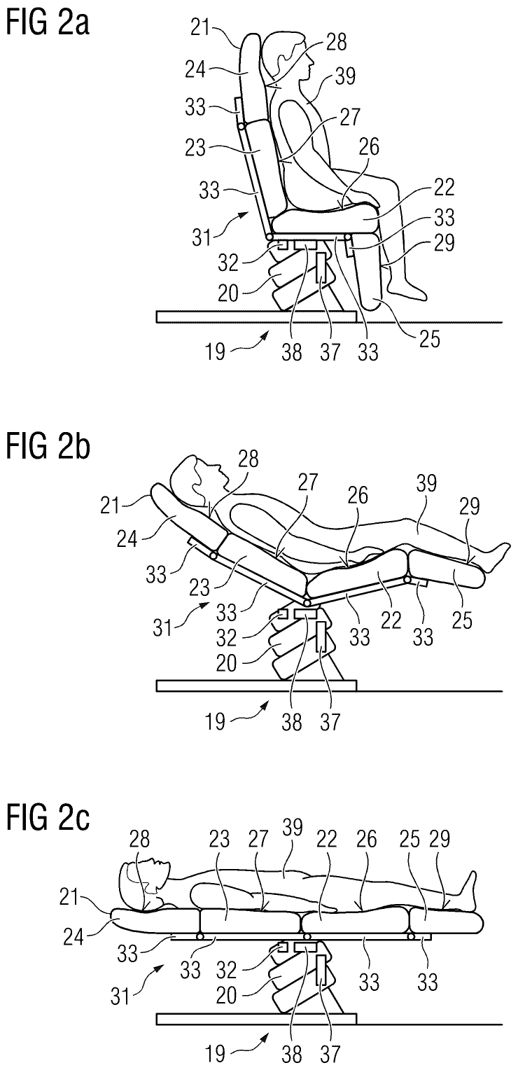

A first chair mode comprises a sitting mode, wherein in the sitting mode, the individual support regions 22, 23, 24, 25 of the patient chair 21 are positioned and/or adjusted in a sitting position by means of the adjusting unit 31 (FIG. 2A). In the sitting position, the different support regions 22, 23, 24, 25 have different orientations of their support areas 26, 27 28, 29. In an embodiment, the support region 22 configured as a seat region of the patient chair 21, e.g. the first support region 22, is herein arranged in a level and/or horizontal position with a level and/or horizontally oriented support area 25. In addition, the support region 23 configured for supporting an upper body of a patient 39, e.g. the second support region 23, is oriented and/or arranged in a substantially perpendicular and/or vertical position with a substantially perpendicularly and/or vertically oriented support area 27. In addition, the further support regions 24, 25, e.g. the support region 24 configured for supporting a head of the patient 39 and/or the support region 25 configured for supporting lower legs and/or feet of a patient 39 are oriented and/or arranged in a substantially perpendicular and/or vertical position with a substantially perpendicularly and/or vertically oriented support area 28, 28. A substantially perpendicular and/or vertical position with a substantially perpendicularly and/or vertically oriented support area 26, 27, 28, 29 can have an inclination angle relative to the level and/or horizontal position with a level and/or horizontally oriented support area 26, 27, 28, 29 of between any suitable range of angle, such as e.g. between 75° and 90°, between 80° and 90°, etc.

In FIGS. 2A to 2C, a transition from the sitting mode (FIG. 2A) of the patient chair 21 into a second chair mode, which is formed by a laying mode (FIG. 2C) of the patient chair 21 is shown. FIG. 2B shows a transition from the sitting mode into the laying mode, wherein in the transition from the sitting mode into the laying mode, the second support region 23, e.g. the support region 23 configured for supporting an upper body of a patient 39, the third support region 24, e.g. the support region 24 configured for supporting a head of a patient 39, and the fourth support region 25, e.g. the support region 25 configured for supporting lower legs and/or feet of the patient 39, are slowly transferred by the adjusting unit 31 from the substantially perpendicular and/or vertical position into a level and/or horizontal position, so that the individual support areas 27, 28, 29 of the support regions 23, 24, 25 all have a level and/or horizontal orientation (FIG. 2C).

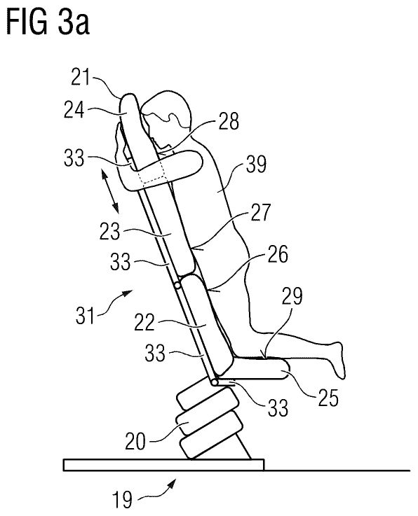

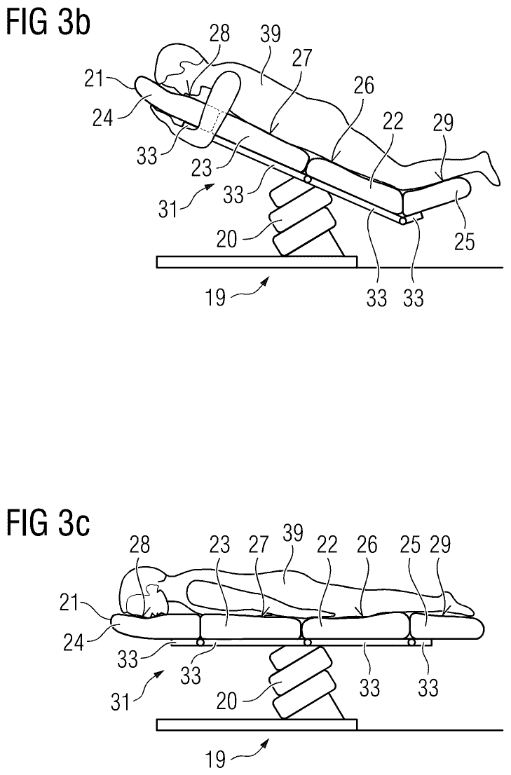

FIG. 3A shows a further chair mode which comprises a prone laying mode. The prone laying mode of the patient chair 21 comprises a defined position and orientation of the individual support regions 22, 23, 24, 25 of the patient chair 21, wherein the different support regions 22, 23, 24, 25 have partially different orientations of their support areas 26, 27 28, 29 in the prone laying mode. The prone laying mode of the patient chair 21 serves to position the patient 39 on the patient chair 21 in a simple manner, wherein the patient 39 abuts the individual support areas 26, 27, 28, 29 with their front side. Following the positioning of the patient 39 on the patient chair 21, the patient chair 21, e.g. the individual support regions 22, 23, 24, 25 of the patient chair 21, is transferred into the laying mode by means of the adjusting unit 31, wherein in the laying position of the patient chair 21, the patient 39 is positioned in a prone laying position on the patient chair 21.

In the prone laying mode, the support region 25 configured for supporting lower legs and/or feet of a patient 39, e.g. the fourth support region 25, is arranged in a level and/or horizontal position with a level and/or horizontally oriented support area 29 so that a patient 39 can kneel thereon. In addition, the remaining support regions 22, 23, 24 of the patient chair 21, e.g. the support region 22 configured as a seat region of the patient chair 21, the support region 23 configured for supporting an upper body of a patient 39 and the support region 24 configured for supporting a head of a patient 39, are arranged in a substantially perpendicular and/or vertical position with a substantially perpendicularly and/or vertically oriented support area 26, 27, 28.

FIG. 3B shows a transition from the prone laying mode into the laying mode, wherein, in the transition from the prone laying mode into the laying mode, the first support region 22, e.g. the seat region, the second support region 23, e.g. the support region 23 configured for supporting an upper body of a patient 39, and the third support region 24, e.g. the support region 24 configured for supporting a head of the patient 39, are slowly transferred by the adjusting unit 31 from the substantially perpendicular and/or vertical position into a level and/or horizontal position, so that the individual support areas 26, 27, 28 of the support regions 22, 23, 24 all have a level and/or horizontal orientation (FIG. 3C).

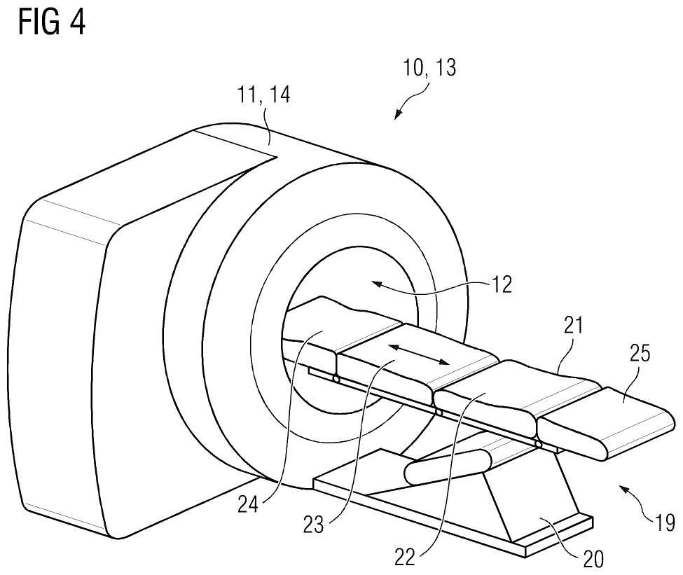

In this laying position, the patient chair 21 is configured such that it is able to be moved into the patient receiving region 12 (FIG. 4).

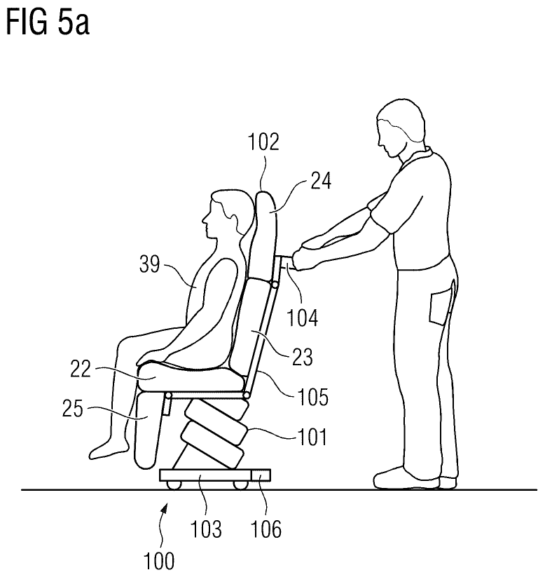

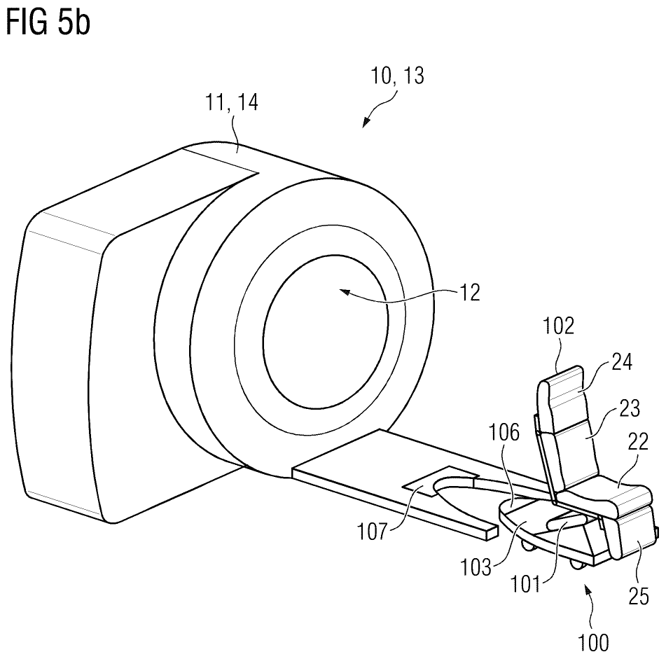

FIGS. 5A and 5B show an alternative exemplary embodiment of a patient support apparatus 100. Components, features, and functions that remain substantially the same are identified with the same reference signs. The following description is essentially restricted to the differences from the exemplary embodiment in FIGS. 1 to 4 wherein, with regard to components, features and functions which remain the same, reference can be made to the description of the exemplary embodiment in FIGS. 1 to 4.

FIGS. 5A and 5B show the patient support apparatus 100 configured as a mobile patient support apparatus 100. The mobile patient support apparatus 100 has a base unit 101 and a patient chair 102. In addition, the patient support apparatus has a traveling unit 103 with a plurality of wheels so that a movement of the patient support apparatus 100 in the room is enabled (FIGS. 5A and 5B).

For guiding and/or moving the patient support apparatus 100 in the room by a user, the patient support apparatus 100 may have a handle unit 104 (FIG. 5A). The handle unit 104 is arranged on a rear side 105 of the patient chair 102, e.g. on a rear side of a second support region 23 of the patient chair 102. On the handle unit 104, e.g. a user-controlled guidance and/or a user-controlled movement of the patient support apparatus 100 can be accomplished, wherein the user-controlled guidance and/or the user-controlled movement of the patient support apparatus 100 can be supported by means of a drive unit (not shown in detail) and a drive control unit (not shown in detail).

The patient support apparatus 100 has a coupling unit 106 for a coupling to the scanner unit 11 of the medical imaging apparatus 10 (FIG. 5B). The coupling unit 106 is herein arranged on the base unit 101 of the patient support apparatus 100. In addition, the scanner unit 11 also has a coupling unit 107, wherein the two coupling units 106, 107 are configured to be compatible with one another. By means of the coupling units 106, 107, both a mechanical coupling and also an electrical coupling and/or a coupling in respect of a data exchange between the patient support apparatus 100 and the scanner unit 11 takes place.

A further embodiment of the patient chair 102 in respect of its support regions 22, 23, 24, 25 and adjustable chair modes corresponds to the embodiments in FIGS. 1 to 4, to which reference is made herewith.

The medical imaging apparatus 10 shown in FIGS. 1 to 5, e.g. the magnetic resonance apparatus 13, can naturally comprise further components that medical imaging apparatuses 10, e.g. magnetic resonance apparatuses 13, typically have. A general mode of operation of a medical imaging apparatus 10, e.g. the magnetic resonance apparatus 13, is also known to a person skilled in the art, so that a detailed description of the further components is not included here.

Although the disclosure has been illustrated and described in detail by way of the exemplary embodiments, the disclosure is not restricted by the examples disclosed and other variations can be derived therefrom by a person skilled in the art without departing from the protective scope of the disclosure.

Independent of the grammatical term usage, individuals with male, female or other gender identities are included within the term.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A medical imaging apparatus, comprising:
a scanner;
a patient receiving region at least partially surrounded by the scanner; and
a patient support comprising a patient chair having two support regions and an adjuster,
wherein a first support region of the two support regions comprises a first support area,
wherein a second support region of the two support regions comprises a second support area, and
wherein the adjuster is configured to adjust an orientation of the first support area and an orientation of the second support area.

2. The medical imaging apparatus as claimed in claim 1, wherein the first support region of the two support regions comprises a seat region of the patient chair, and
wherein the second support region of the two support regions is configured to support an upper body of a patient.

3. The medical imaging apparatus as claimed in claim 1, wherein:
the patient chair comprises a further support region configured to support a head of a patient,
the further support region comprises a further support area, and
the adjuster is configured to adjust an orientation of the further support area.

4. The medical imaging apparatus as claimed in claim 1, wherein:
the patient chair comprises a further support region,
the further support region is configured to support lower legs and/or feet of a patient,
the further support region comprises a further support area, and
the adjuster is configured to adjust an orientation of the further support area.

5. The medical imaging apparatus as claimed in claim 1, wherein the patient chair is configured to provide a laying mode in which the adjuster positions the two support regions of the patient chair into a laying position, and the patient chair is movable into the patient receiving region.

6. The medical imaging apparatus as claimed in claim 1, wherein the patient chair is configured to provide a sitting mode in which the adjuster positions the two support regions of the patient chair into a sitting position.

7. The medical imaging apparatus as claimed in claim 1, wherein the patient chair comprises a prone laying mode in which the adjuster positions the two support regions of the patient chair into a prone laying position.

8. The medical imaging apparatus as claimed in claim 1, wherein the adjuster comprises a controller configured to perform separate actuation of the two support regions for adjusting an orientation of the first and the second support area, respectively.

9. The medical imaging apparatus as claimed in claim 1, wherein the patient support comprises a base having a vertical adjuster, and
wherein the vertical adjuster is configured to adjust a vertical position of the patient chair.

10. The medical imaging apparatus as claimed in claim 1, wherein the patient support comprises a rotator configured to adjust an orientation of the patient chair relative to the scanner and/or a base of the patient support.

11. The medical imaging apparatus as claimed in claim 1, wherein one support region of the two support regions of the patient support comprises a receiving region configured to receive an accessory.

12. The medical imaging apparatus as claimed in claim 1, wherein the patient support comprises a stationary patient support having a fixed position relative to the scanner.

13. The medical imaging apparatus as claimed in claim 1, wherein the patient support comprises a mobile patient support having a coupler that is compatible with a corresponding coupler of the scanner.

14. The medical imaging apparatus as claimed in claim 1, further comprising:
a magnetic resonance apparatus that is compatible with the patient support.
